(19) United States
(12) Patent Application Publication
(10) Pub. No.: US 2016/0108004 A1
(43) Pub. Date: Apr. 21, 2016

(54) TREATMENT OR PREVENTION OF NEURODEGENERATIVE DISORDERS USING MENTHOL, LINALOOL AND/OR ICILIN

(71) Applicant: NESTEC S.A., Vevey (CH)

(72) Inventors: Susana CAMACHO, Lausanne (CH); Stephanie MICHLIG=GONZALEZ, Le Mont-sur-Lausanne (CH); Johannes LE COUTRE, Pully (CH); Henry MARKRAM, Lausanne (CH); Maurizio PEZZOLI, Lausanne (CH)

(21) Appl. No.: 14/892,004

(22) PCT Filed: May 23, 2014
(86) PCT No.: PCT/EP2014/060632

§ 371 Date: Nov. 18, 2015

Related U.S. Application Data
(60) Provisional application No. 61/827,243, filed on May 24, 2013.

(51) Int. Cl. C07D 239/36; C07C 33/02; C07C 35/12

(52) U.S. Cl. CPC C07D 239/36; C07C 35/12; C07C 33/02; A23L 1/30; A23V 2002/00

(57) ABSTRACT

Compositions for treatment or prevention of neurodegenerative disorders are provided, and the compositions contain a therapeutically effective amount of a compound selected from the group consisting of Menthol, Linalool, Icilin and combinations thereof. Methods for treatment or prevention of neurodegenerative disorders are also provided, and the methods include administering such compositions.

### BACKGROUND

The present disclosure generally relates to methods and compositions for prevention or treatment of neurodegenerative disorders. More specifically, the present disclosure relates to compositions comprising at least one of Menthol, Linalool or Icilin and further relates to methods comprising administering such compositions.

Neurodegenerative disorders are characterized by a progressive loss of structure and function of neurons, ultimately leading to death of neurons. In many diseases, such as Alzheimer's disease, Parkinson's disease and Huntington's disease, neurodegenerative processes are a major detrimental component, modulating the course of disease.

The biggest risk factor for neurodegenerative diseases is aging. Many of these diseases are late-onset, meaning that there are some factors that change as a person gets older. One constant factor is that in each disease, neurons gradually lose function as the disease progresses with age. A further consequence of such continuous and severe loss of neuronal function is the loss of the cognitive ability as can be manifested in different forms of dementia. Thereby, normal cognitive functions can be affected with, for example, a loss of memory, attention or mental concentration, language, and the ability to solve problems. Especially in the later stages of a neurodegenerative condition, affected persons may be disoriented in time, in place, and in person. Neurodegenerative disorders, though often treatable to some degree, are usually due to causes that are progressive and incurable.

One of the main causes for neurodegenerative processes is excitotoxicity, the pathological process by which nerve cells are damaged and killed through excessive stimulation by neurotransmitters such as glutamate, among several other causes such as increased levels of inoxidative stress, mitochondrial dysfunction, inflammatory changes, iron accumulation, and protein aggregation. Glutamate antagonists are common neuroprotective treatments. These antagonists inhibit the binding of glutamate to NMDA receptors such that accumulation of Ca²⁺ and therefore excitotoxicity can be avoided. However, use of glutamate antagonists presents a huge obstacle because the treatment interferes with the normal action of glutamate under standard conditions. A number of glutamate antagonists have been explored as options in central nervous system (CNS) disorders, but many are found to lack efficacy or have intolerable side effects.

There is a clear and persisting need to prevent and treat neurodegenerative disorders, particularly for the aging population.

### SUMMARY

The present inventors surprisingly and unexpectedly found that several active compounds from spices can depress neural activity in the neocortex and the amygdale. These compounds are Menthol and Linanool which are transient receptor potential M8 (TRPM8) channel agonists. The present inventors discovered the same effect with Icilin, a synthetic super-agonist of the TRPM8 ion channel, even though the structure of Icilin is not related to Menthol.

Accordingly, in a general embodiment, the present disclosure provides a method for treating a neurodegenerative disorder. The method comprises administering to an individual having the neurodegenerative disorder a composition comprising a therapeutically effective amount of a compound selected from the group consisting of Menthol, Linalool, Icilin and combinations thereof.

In an embodiment, the neurodegenerative disorder is selected from the group consisting of Alzheimer's disease, other dementias, degenerative nerve diseases, genetic brain disorders, Parkinson's disease, amyotrophic lateral sclerosis, Huntington's disease, prion diseases and combinations thereof.

In a related embodiment, the composition is selected from the group consisting of a medicament, a food product and a supplement to a food product.

In a related embodiment, the composition is administered periodically for at least one year.

In another embodiment, a method for preventing a neurodegenerative disorder is provided. The method comprises administering to an individual a composition comprising a therapeutically effective amount of a compound selected from the group consisting of menthol, linalool, icilin and combinations thereof.

In a related embodiment, the neurodegenerative disorder is selected from the group consisting of Alzheimer's disease, other dementias, degenerative nerve diseases, genetic brain disorders, Parkinson's disease, amyotrophic lateral sclerosis, Huntington's disease, prion diseases and combinations thereof.

In a related embodiment, the individual is an aging human.

In a related embodiment, the individual is an elderly human.

In a related embodiment, the composition is administered periodically for at least one year. The composition can be administered daily.

In another embodiment, a composition for treating or preventing a neurodegenerative disorder is provided. The composition comprises a therapeutically effective amount of a compound selected from the group consisting of Menthol, Linalool, Icilin and combinations thereof.

In a related embodiment, the composition is a medicament.

In a related embodiment, the composition is a food product. The food product can comprise a component selected from the group consisting of protein, carbohydrate, fat and combinations thereof.

In a related embodiment, the composition is a supplement to a food product.

An advantage of the present disclosure is to prevent or treat neurodegenerative disorders more effectively and/or more safely than glutamate antagonists.

Another advantage of the present disclosure is to prevent or treat neurodegenerative disorders without interfering with the normal action of glutamate under standard conditions.

Still another advantage of the present disclosure is to prevent or treat neurodegenerative disorders with compounds that can be easily and safely used in food products.

Yet another advantage of the present disclosure is to prevent or treat neurodegenerative disorders by targeting the pre-synaptic phase of neuronal firing.

An additional advantage of the present disclosure is to prevent or treat neurodegenerative disorders by targeting the pre-synaptic phase of neuronal firing while reducing the possibility of excitotoxicity.

Another advantage of the present disclosure is to prevent or treat neurodegenerative disorders with naturally-occurring compounds that can be found in spices.

Still another advantage of the present disclosure is to prevent or treat neurodegenerative disorders with tolerable side effects or no side effects.

Additional features and advantages are described herein, and will be apparent from, the following Detailed Description and the Figures.

### BRIEF DESCRIPTION OF THE FIGURES

FIG. 1 shows the chemical structures of compounds that can be used in embodiments of the composition according to the present disclosure.

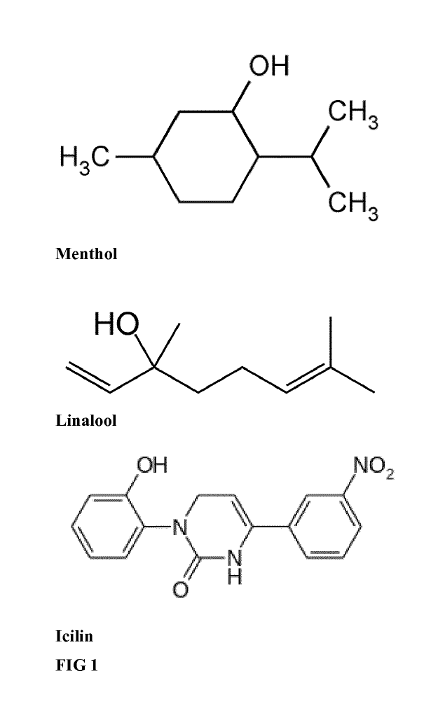

FIG. 2 shows charts of whole cell, current clamp recordings in a Lateral Amygdala glutamatergic neuron (in a mouse brain slice) in the absence (control) and presence of Linalool, Icilin or Menthol.

FIG. 3 shows a chart of whole cell, current clamp recordings in a Lateral Amygdala glutamatergic neuron (in a mouse brain slice) with increasing concentration of gabazine applied extracellularly during recordings of 5 min each (washout 10 min)

FIG. 4 shows a chart of whole cell, current clamp recordings in a Lateral Amygdala glutamatergic neuron (in a mouse brain slice) showing enhanced detail of a burst.

FIG. 5 shows a chart of whole cell, current clamp recordings in a Lateral Amygdala glutamatergic neuron (in a mouse brain slice) with increasing concentration of gabazine applied extracellularly during recordings of 5 min. each (washout 10 min.) while 10 minutes previous to and during the exposure of the different concentrations of gabazine, 250 μM menthol was also applied extracellularly.

### DETAILED DESCRIPTION

All percentages expressed herein are by weight of the total weight of the composition unless expressed otherwise. When reference is made to the pH, values correspond to pH measured at 25° C. with standard equipment. As used in this disclosure and the appended claims, the singular forms “a,” “an” and “the” include plural referents unless the context clearly dictates otherwise. As used herein, “about” is understood to refer to numbers in a range of numerals. Moreover, all numerical ranges herein should be understood to include all integers, whole or fractions, within the range. The food composition disclosed herein may lack any element that is not specifically disclosed herein. Thus, “comprising” includes “consisting essentially of” and “consisting of.”

As used herein, “neurodegenerative disorders” are hereditary or sporadic conditions which are characterized by progressive nervous system dysfunction. These disorders are often associated with atrophy of the affected central or peripheral structures of the nervous system. Non-limiting examples of neurodegenerative disorders include Alzheimer's disease and other dementias, degenerative nerve diseases, genetic brain disorders, Parkinson's disease, amyotrophic lateral sclerosis (ALS or Lou Gehrig's disease), Huntington's disease, and prion diseases.

“Prevention” includes reduction of risk and/or severity of neurodegenerative disorders. The terms “treatment,” “treat” and “to alleviate” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The term does not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment,” “treat” and “to alleviate” are also intended to include the potentiation or otherwise enhancement of one or more primary prophylactic or therapeutic measure. The terms “treatment,” “treat” and “to alleviate” are further intended to include the dietary management of a disease or condition or the dietary management for prophylaxis or prevention a disease or condition. A treatment can be patient- or doctor-related.

As used herein, a “therapeutically effective amount” is an amount that prevents a deficiency, treats a disease or medical condition in an individual or, more generally, reduces symptoms, manages progression of the diseases or provides a nutritional, physiological, or medical benefit to the individual.

“Animal” includes, but is not limited to, mammals, which includes but is not limited to, rodents, aquatic mammals, domestic animals such as dogs and cats, farm animals such as sheep, pigs, cows and horses, and humans. Where “animal,” “mammal” or a plural thereof is used, these terms also apply to any animal that is capable of the effect exhibited or intended to be exhibited by the context of the passage. As used herein, the term “patient” is understood to include an animal, especially a mammal, and more especially a human that is receiving or intended to receive treatment, as treatment is herein defined. While the terms “individual” and “patient” are often used herein to refer to a human, the present disclosure is not so limited. Accordingly, the terms “individual” and “patient” refer to any animal, mammal or human, having or at risk for a medical condition that can benefit from the treatment.

An “aging” animal has exceeded 50% of the average lifespan for its particular species and/or breed within a species. An animal is considered “elderly” if it has surpassed the first two thirds of the average expected lifespan in its country of origin, preferably if it has surpassed the first three quarters of the average expected lifespan in its country of origin, more preferably if it has surpassed the first four fifths of the average expected lifespan in its country of origin. An “elderly human” means a person with a chronological age of 65 years or older.

“Food product” and “food composition,” as used herein, are understood to include any number of optional additional ingredients, including conventional food additives, for example one or more proteins, carbohydrates, fats, acidulants, thickeners, buffers or agents for pH adjustment, chelating agents, colorants, emulsifiers, excipients, flavor agents, minerals, osmotic agents, a pharmaceutically acceptable carrier, preservatives, stabilizers, sugars, sweeteners, texturizers and/or vitamins. The optional ingredients can be added in any suitable amount.

As set forth above, the present inventors surprisingly and unexpectedly found that several active compounds from spices can depress neural activity in neocortex and amygdale. These compounds are Menthol and Linanool which are transient receptor potential M8 (TRPM8) channel agonists. The present inventors discovered the same effect with Icilin, a synthetic super-agonist of the TRPM8 ion channel, even though the structure of Icilin is not related with Menthol; nevertheless, Icilin produces an extreme sensation of cold both in humans and animals. These natural compounds reduce neural excitability by 1) increasing the threshold to trigger an action potential and consequently increasing the amount of current required to trigger an action potential in the neocortex; and 2) abortion of action potentials at higher stimulation levels, most likely related to the use-dependent block of Na+ channels in the neocortex and lateral amygdale. These active compounds change the firing patterns especially at higher stimulation levels where a progressive and dramatic reduction of the action potential (APs) amplitude occurs until complete abortion of APs.

Without wishing to be bound by theory, the inventors believe that the mechanism underlying the selected active compounds of spices, namely Menthol, Linanool and Icilin, solves two main problems compared to neuroprotective glutamate antagonists: 1) Menthol, Linanool and Icilin target a presynaptic phase of APs, decreasing activity and diminishing glutamate release, which reduces drastically the possibility of reaching excitotoxicity levels; and 2) Menthol, Linanool and Icilin act stronger in the high stimulation context. In contrast to glutamate antagonists that typically inhibit the binding of glutamate to NMDA receptors, Menthol, Linanool and Icilin decrease neuronal activity, and target the pre-synaptic phase of the firing to reduce the possibilities of excitotoxicity one step earlier.

Accordingly, the composition provided by the present disclosure comprises a therapeutically effective amount of at least one of Menthol, Linalool or Icilin. In an embodiment, a neurodegenerative disorder is treated or prevented by administering to an individual in need of same the composition comprising at least one of Menthol, Linalool or Icilin. For example, the composition comprising at least one of Menthol, Linalool or Icilin can be administered to an individual having a neurodegenerative disorder to treat the neurodegenerative disorder. The neurodegenerative disorder can be Alzheimer's disease, other dementias, degenerative nerve diseases, genetic brain disorders, Parkinson's disease, amyotrophic lateral sclerosis, Huntington's disease, prion diseases and combinations thereof.

The composition comprising at least one of Menthol, Linalool or Icilin may be a medicament, a food product or a supplement to a food product. The supplement may be in the form of tablets, capsules, pastilles or a liquid, for example. The supplement may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins or the like), adsorbents, carriers, fillers, co-compounds, dispersing agents, wetting agents, processing aids (solvents), flowing agents, taste masking agents, weighting agents, jellifying agents and gel forming agents. The supplement may also contain conventional pharmaceutical additives and adjuvants, excipients and diluents, including, but not limited to, water, gelatin of any origin, vegetable gums, ligninsulfonate, talc, sugars, starch, gum arabic, vegetable oils, polyalkylene glycols, flavoring agents, preservatives, stabilizers, emulsifying agents, buffers, lubricants, colorants, wetting agents, fillers, and the like.

The supplement can be added in a product acceptable to the consumer as an ingestible carrier or support. Non-limiting examples of such carriers or supports are a pharmaceutical, a food composition, and a pet food composition. Non-limiting examples for food and pet food compositions are milks, yogurts, curds, cheeses, fermented milks, milk-based fermented products, fermented cereal based products, milk-based powders, human milks, preterm formulas, infant formulas, oral supplements, and tube feedings.

In an embodiment, the composition comprising at least one of Menthol, Linalool or Icilin is administered to a human, preferably an adult human being. Many of the neurodegenerative disorders or cognitive dysfunctions occur with the progression of age of an individual. Clinical manifestation is therefore often only perceived in adulthood or at an already advanced age. Hence, the composition is preferably administered to adult persons, while or before the onset of such a neurodegenerative disorder. Thereby, advantageously, the neurodegenerative disorder is treated early on to limit or reduce the further progression of the degeneration of neuronal cells. Ideally, the onset of such degeneration can be delayed or reduced due to a preventive effect of an early application in adulthood, when the individual is still healthy and in full cognitive capacity.

In an alternative embodiment, the composition comprising at least one of Menthol, Linalool or Icilin is administered to a non-human animal, preferably a cat or a dog. Similarly to humans, neurodegeneration can be observed with animals, in particular with farm animals and animals kept as pets. Thereby, it is particularly difficult for an owner of a cat or a dog to see their dear companion animal affected by a neurodegenerative disorder with the progression of the age of the animal. Advantageously, the composition comprising at least one of Menthol, Linalool or Icilin can be provided to a companion animal by its owner.

The composition comprising at least one of Menthol, Linalool or Icilin is preferably intended for a consumption regime over an extended period of time, preferably over several years. For example, the composition can be administered periodically, such as weekly or daily, for at least one year, preferably at least two years, and more preferably even longer amounts of time. Neurodegenerative disorders are slow processes, which can occur only gradually, but progressively over many years and ultimately may lead to the death of an affected individual. Typically, persons affected with such a degenerative disorder, depending on the nature of which disease, may be affected and survive for 5, 10, 15 or 20 years or longer. Therefore, the composition can be used for the entirety of such period or preferably starting before the onset of such a disorder by an individual.

Each of Menthol, Linalool and/or Icilin can be administered to the individual in a daily amount of 0.0015 mg/kg of body weight to 400 mg/kg of body weight, preferably 0.1 mg/kg of body weight to 300 mg/kg of body weight, more preferably 1.0 mg/kg of body weight to 200 mg/kg of body weight, and most preferably 10.0 mg/kg of body weight to 100 mg/kg of body weight. For example, each of Menthol, Linalool and/or Icilin can be administered to the individual in a daily amount of 0.0015 mg/kg of body weight to 0.01 mg/kg of body weight, 0.01 mg/kg of body weight to 0.1 mg/kg of body weight, 0.1 mg/kg of body weight to 1.0 mg/kg of body weight, 1.0 mg/kg of body weight to 10.0 mg/kg of body weight, 10.0 mg/kg of body weight to 100.0 mg/kg of body weight, 100.0 mg/kg of body weight to 200.0 mg/kg of body weight, 200.0 mg/kg of body weight to 300.0 mg/kg of body weight, or 300.0 mg/kg of body weight to 400.0 mg/kg of body weight.

### EXAMPLES

The following non-limiting examples present scientific data developing and supporting the concept of treatment or prevention of neurodegenerative disorders using Menthol, Linalool and Icilin.

A mouse brain slice was used to study the effects of Menthol, Linalool and Icilin. The amygdaloid complex is located within the medial temporal lobe in neocortex and amygdala. The lateral and basolateral nuclei of the amygdaloid complex receive sensory information from cortical and thalamic structures, process the information, and then transmit the information, either directly or through the basal nucleus, to the central nucleus. For experimental analysis of neural activity, synaptic responses from the basolateral complex can be evoked electrically using electrodes, and the action potentials can be measured.

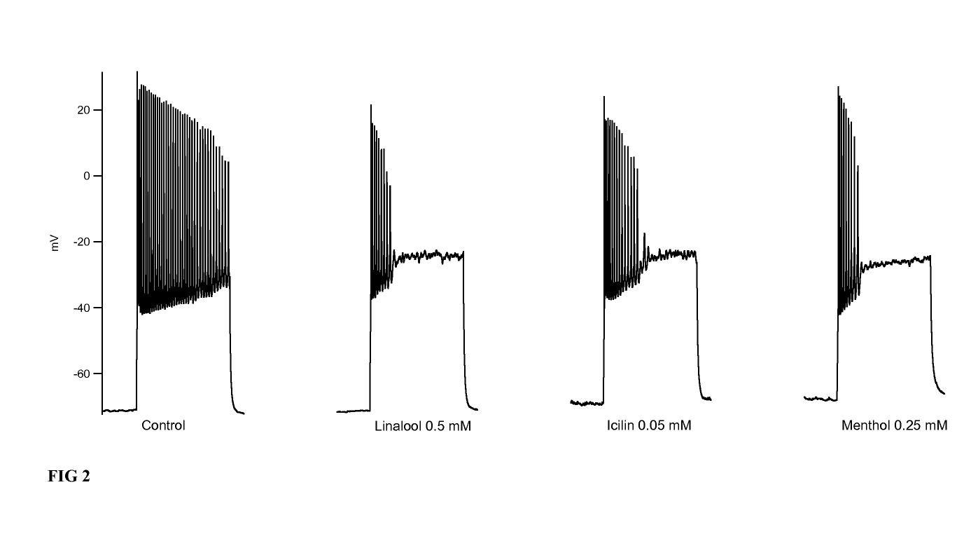

FIG. 2 shows recordings in the absence of Menthol, Linalool or Icilin (control) and recordings in the presence of Menthol, Linalool or Icilin. A square pulse of 2.5 s was applied at high depolarization of membrane potential (approximately −30 mV). The recordings show that, in the presence of the TRPM8 ligands at high depolarization levels, inactivation of the sodium fast channels happens sooner relative to control, avoiding further neural firing.

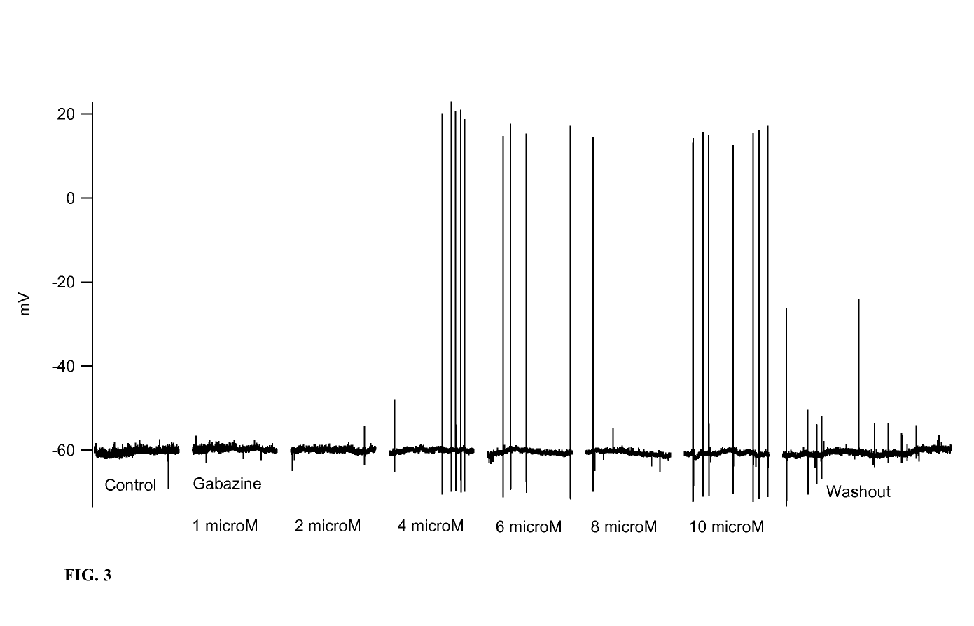

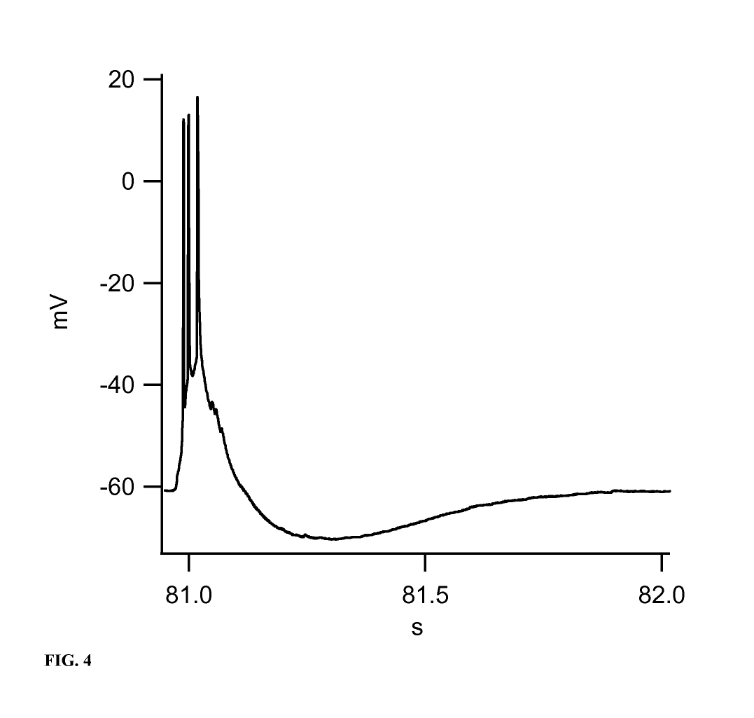

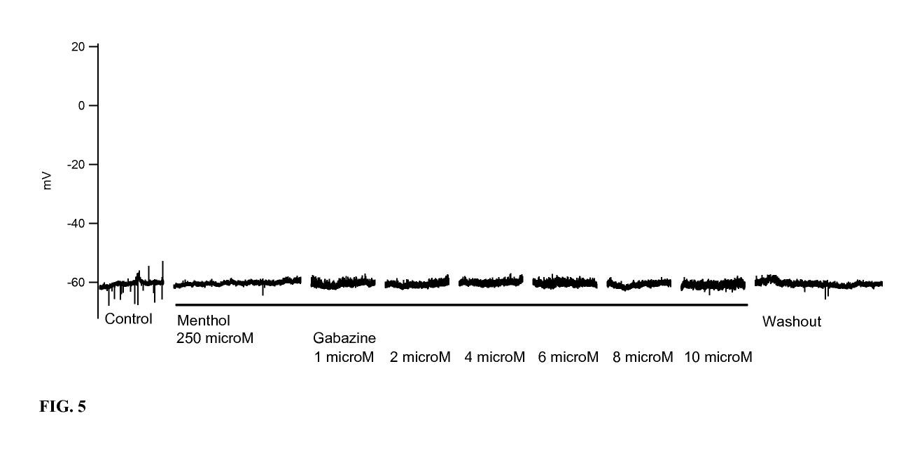

FIG. 3 shows recordings in increasing concentrations of gabazine, a GABA A blocker, applied extracellularly during recordings of 5 minutes each with 10 minute washout. As shown, neurons spontaneously present action potential bursts due to massive presynaptic discharges. FIG. 4 depicts enhanced detail of one of the bursts and shows that serial action potentials can be observed in a single burst. For comparison to FIG. 3, FIG. 5 shows recordings under the same conditions, namely increasing concentrations of gabazine applied extracellularly during recordings of 5 minutes each with 10 minute washout, except that in FIG. 5, Menthol 250 μM was applied extracellularly at 10 minutes previous to and during the exposure of the different concentrations of gabazine. As illustrated in the figure, neurons show a complete absence or a strongly decreased presence of spontaneous bursts (compare FIG. 5 to FIG. 3).

These experimental results demonstrate that Menthol, Linalool and Icilin increase the threshold to trigger an action potential and consequently increase the amount of current required to trigger an action potential in the neocortex, and also abort action potentials at higher stimulation levels.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method for treating a neurodegenerative disorder comprising administering to an individual having the neurodegenerative disorder a composition comprising a therapeutically effective amount of a compound selected from the group consisting of Menthol, Linalool, Icilin and combinations thereof.

2. The method of claim 1 wherein the neurodegenerative disorder is selected from the group consisting of Alzheimer's disease, other dementias, degenerative nerve diseases, genetic brain disorders, Parkinson's disease, amyotrophic lateral sclerosis, Huntington's disease, prion diseases and combinations thereof.

3. The method of claim 1 wherein the composition is selected from the group consisting of a medicament, a food product and a supplement to a food product.

4. The method of claim 1 wherein the composition is administered periodically for at least one year.

5. A method for preventing a neurodegenerative disorder comprising administering to an individual a composition comprising a therapeutically effective amount of a compound selected from the group consisting of Menthol, Linalool, Icilin and combinations thereof.

6. The method of claim 5 wherein the neurodegenerative disorder is selected from the group consisting of Alzheimer's disease, other dementias, degenerative nerve diseases, genetic brain disorders, Parkinson's disease, amyotrophic lateral sclerosis, Huntington's disease, prion diseases and combinations thereof.

7. The method of claim 5 wherein the individual is an aging human.

8. The method of claim 5 wherein the individual is an elderly human.

9. The method of claim 5 wherein the composition is administered periodically for at least one year.

10. The method of claim 5 wherein the composition is administered daily.

11. A composition for treating or preventing a neurodegenerative disorder comprising a therapeutically effective amount of a compound selected from the group consisting of Menthol, Linalool, Icilin and combinations thereof.

12. The composition of claim 11 wherein the composition is a medicament.

13. The composition of claim 11 wherein the composition is a food product.

14. The composition of claim 13 wherein the food product comprises a component selected from the group consisting of protein, carbohydrate, fat and combinations thereof.

15. The composition of claim 11 wherein the composition is a supplement to a food product.
